(19) United States
(12) Patent Application Publication
(10) Pub. No.: US 2012/0010528 A1
(43) Pub. Date: Jan. 12, 2012

(54) Systems and methods for predicting disease progression in patients treated with radiotherapy

(75) Inventors: Michael Donovan, (US); Faisal Khan, (US)

(73) Assignee: Aureon Biosciences, Inc., Yonkers, NY (US)

(21) Appl. No.: 13/066,870

(22) Filed: Apr. 26, 2011

Related U.S. Application Data
(60) Provisional application No. 61/343,306, filed on Apr. 26, 2010.

(51) Int. Cl. A61B 10/02; G06K 9/00

(52) U.S. Cl. 600/567; 382/128

(57) ABSTRACT

Clinical information, molecular information and/or computer-generated morphometric information is used in a predictive model for predicting the occurrence of a medical condition. In an embodiment, a model predicts whether a disease (e.g., prostate cancer) is likely to progress in a patient after radiation therapy. In some embodiments, the molecular and computer-generated morphometric information is obtained through computer analysis of tissue obtained from the patient via a needle biopsy at diagnosis and before treatment of the patent with radiation therapy.

### CROSS-REFERENCE TO RELATED APPLICATION

This application claims priority to U.S. Provisional Application No. 61/343,306, filed Apr. 26, 2010, which is hereby incorporated by reference herein in its entirety.

### FIELD OF THE INVENTION

Embodiments of the present invention relate to methods and systems for predicting the occurrence of a medical condition such as, for example, the presence, indolence, recurrence, or progression of disease (e.g., cancer), responsiveness or unresponsiveness to a treatment for the medical condition, or other outcome with respect to the medical condition. For example, in some embodiments of the present invention, systems and methods are provided that use clinical information, molecular information, and/or computer-generated morphometric information in a predictive model that predicts, at the time of diagnosis of cancer (e.g., prostate cancer) in a patient, the likelihood of disease progression in the patient even if the patient is treated with primary radiotherapy. In some embodiments, some or all of the information evaluated by these systems and methods is generated from, or otherwise available at the time of, a needle biopsy of tissue from the patient.

### BACKGROUND OF THE INVENTION

Physicians are required to make many medical decisions ranging from, for example, whether and when a patient is likely to experience a medical condition to how a patient should be treated once the patient has been diagnosed with the condition. Determining an appropriate course of treatment for a patient may increase the patient's chances for, for example, survival, recovery, and/or improved quality of life. Predicting the occurrence of an event also allows individuals to plan for the event. For example, predicting whether a patient is likely to experience occurrence (e.g., presence, recurrence, or progression) of a disease may allow a physician to recommend an appropriate course of treatment for that patient.

When a patient is diagnosed with a medical condition, deciding on the most appropriate therapy is often confusing for the patient and the physician, especially when no single option has been identified as superior for overall survival and quality of life. Traditionally, physicians rely heavily on their expertise and training to treat, diagnose, and predict the occurrence of medical conditions. For example, pathologists use the Gleason scoring system to evaluate the level of advancement and aggression of prostate cancer, in which cancer is graded based on the appearance of prostate tissue under a microscope as perceived by a physician. Higher Gleason scores are given to samples of prostate tissue that are more undifferentiated. Although Gleason grading is widely considered by pathologists to be reliable, it is a subjective scoring system. Particularly, different pathologists viewing the same tissue samples may make conflicting interpretations.

It is believed by the present inventors that more accurate, stable, and comprehensive approaches to predicting the occurrence of medical conditions are needed.

In view of the foregoing, it would be desirable to provide systems and methods for treating, diagnosing, and predicting the occurrence of medical conditions, responses, and other medical phenomena with improved predictive power. For example, it would be desirable to provide systems and methods for predicting, at the time of diagnosis of cancer (e.g., prostate cancer) in a patient, the likelihood of disease progression in the patient even if the patient is treated with radiation therapy.

### SUMMARY OF THE INVENTION

Embodiments of the present invention provide automated systems and methods for predicting the occurrence of medical conditions. As used herein, predicting an occurrence of a medical condition may include, for example, predicting whether and/or when a patient will experience an occurrence (e.g., presence, recurrence or progression) of disease such as cancer, predicting whether a patient is likely to respond to one or more therapies (e.g., a new pharmaceutical drug), or predicting any other suitable outcome with respect to the medical condition. Predictions by embodiments of the present invention may be used by physicians or other individuals, for example, to select an appropriate course of treatment for a patient, diagnose a medical condition in the patient, and/or predict the risk of disease progression in the patient.

In some embodiments of the present invention, systems, apparatuses, methods, and computer readable media are provided that use clinical information, molecular information and/or computer-generated morphometric information in a predictive model for predicting the occurrence of a medical condition. For example, a predictive model according to some embodiments of the present invention may be provided which is based on one or more of the features listed in FIGS. 5 and 6, Tables 2, 3, and 4, and/or other features.

For example, in an embodiment, a predictive model is provided that predicts whether a disease (e.g., prostate cancer) is likely to progress in a patient even after radiation therapy, where the model is based on one or more clinical features, one or more molecular features, and/or one or more computer-generated morphometric features generated from one or more tissue images. For example, in some embodiments, the model may be based on one or more (e.g., all) of the features listed in FIGS. 5 and 6, Tables 2, 3, and 4, and optionally other features. Such features include, for example, one or more (e.g., all) of: pre-operative PSA; Gleason score; a morphometric measurement of lumens derived from a tissue image (e.g., median are of lumens); a morphometric measurement of epithelial nuclei derived from a tissue image (e.g., relative area of epithelial nuclei relative to total tumor area); a molecular measurement of Ki67-positive epithelial nuclei (e.g., relative area of Ki67-positive epithelial nuclei to the total area of epithelial nuclei, or relative area of Ki67-positive epithelial nuclei to area of tumor); and/or other features.

In another embodiment of the present invention, the predicative model may be based on features including one or more (e.g., all) of: preoperative PSA; dominant Gleason Grade; Gleason Score; at least one of a measurement of expression of androgen receptor (AR) in epithelial and/or stromal nuclei (e.g., tumor epithelial and/or stromal nuclei) and a measurement of expression of Ki67-positive epithelial nuclei (e.g., tumor epithelial nuclei); a morphometric measurement of average edge length in the minimum spanning tree (MST) of epithelial nuclei; and a morphometric measurement of area of non-lumen associated epithelial cells relative to total tumor area. In some embodiments, the dominant Gleason Grade comprises a dominant biopsy Gleason Grade. In some embodiments, the Gleason Score comprises a biopsy Gleason Score. In some embodiments, such a model may be used to predict whether a disease (e.g., prostate cancer) is likely to progress in a patient even after radiation therapy.

In some embodiments of the present invention, computer-generated morphometric features may be generated based on computer analysis of one or more images of tissue subject to staining with hematoxylin and eosin (H&E). In some embodiments of the present invention, computer-generated morphometric features and/or molecular features may be generated from computer analysis of one or more images of tissue subject to multiplex immunofluorescence (IF).

In still another aspect of embodiments of the present invention, a test kit is provided for treating, diagnosing and/or predicting the occurrence of a medical condition. Such a test kit may be situated in a hospital, other medical facility, or any other suitable location. The test kit may receive data for a patient (e.g., including clinical data, molecular data, and/or computer-generated morphometric data), compare the patient's data to a predictive model (e.g., programmed in memory of the test kit) and output the results of the comparison. In some embodiments, the molecular data and/or the computer-generated morphometric data may be at least partially generated by the test kit. For example, the molecular data may be generated by an analytical approach subsequent to receipt of a tissue sample for a patient. The morphometric data may be generated by segmenting an electronic image of the tissue sample into one or more objects, classifying the one or more objects into one or more object classes (e.g., epithelial nuclei, epithelial cytoplasm, stroma, lumen, red blood cells, etc.), and determining the morphometric data by taking one or more measurements for the one or more object classes. In some embodiments, the test kit may include an input for receiving, for example, updates to the predictive model. In some embodiments, the test kit may include an output for, for example, transmitting data, such as data useful for patient billing and/or tracking of usage, to another device or location.

### BRIEF DESCRIPTION OF THE DRAWINGS

For a better understanding of embodiments of the present invention, reference is made to the following detailed description, taken in conjunction with the accompanying drawings, in which like reference characters refer to like parts throughout, and in which:

FIGS. 1A and 1B are block diagrams of systems that use a predictive model to treat, diagnose or predict the occurrence of a medical condition according to some embodiments of the present invention;

FIG. 1C is a block diagram of a system for generating a predictive model according to some embodiments of the present invention;

FIG. 2 is a flowchart of illustrative stages involved in image segmentation and object classification in, for example, digitized images of H&E-stained tissue according to some embodiments of the present invention;

FIG. 3A is an image of prostate tissue obtained via a needle biopsy and subject to staining with hematoxylin and eosin (H&E) according to some embodiments of the present invention;

FIG. 3B is a segmented and classified version of the image in FIG. 4A according to some embodiments of the present invention, in which gland unit objects are formed from seed lumen, epithelial nuclei, and epithelial cytoplasm, and in which isolated/non-gland-associated tumor epithelial cells are also identified in the image;

FIG. 4A is an image of tissue subject to multiplex immunofluorescence (IF) in accordance with some embodiments of the present invention;

FIG. 4B shows a segmented and classified version of the image in FIG. 4A, in which the objects epithelial nuclei, cytoplasm, and stroma nuclei have been identified according to some embodiments of the present invention;

FIG. 5 is a listing of clinical and computer-generated morphometric features used by a model to predict whether a disease is likely to progress in a patient even after radiation therapy according to an embodiment of the present invention; and

FIG. 6 is a listing of molecular and computer-generated morphometric features used by a model to predict whether a disease is likely to progress in a patient even after radiation therapy according to an embodiment of the present invention.

### DETAILED DESCRIPTION OF THE INVENTION

Embodiments of the present invention relate to methods and systems that use computer-generated morphometric information, clinical information, and/or molecular information in a predictive model for predicting the occurrence of a medical condition. For example, in some embodiments of the present invention, clinical, molecular, and computer-generated morphometric information are used to predict whether or not a disease (e.g., prostate cancer) is likely to progress in a patient even after radiation therapy. In some embodiments, a predictive model outputs a value indicative of such a prediction based on information available at the time of diagnosis of the disease in the patient. For example, some or all of the information evaluated by the predictive model may be generated from, or otherwise available at the time of, a needle biopsy from the patient. In other embodiments, the teachings provided herein are used to predict the occurrence (e.g., presence, recurrence, or progression) of other medical conditions such as, for example, other types of disease (e.g., epithelial and mixed-neoplasms including breast, colon, lung, bladder, liver, pancreas, renal cell, and soft tissue) and the responsiveness or unresponsiveness of a patient to one or more therapies (e.g., pharmaceutical drugs). These predictions may be used by physicians or other individuals, for example, to select an appropriate course of treatment for a patient, diagnose a medical condition in the patient, and/or predict the risk or likelihood of disease progression in the patient.

In an aspect of the present invention, an analytical tool such as, for example, a module configured to perform support vector regression for censored data (SVRc), a support vector machine (SVM), and/or a neural network may be provided that determines correlations between clinical features, molecular features, computer-generated morphometric features, combinations of such features, and/or other features and a medical condition. The correlated features may form a model that can be used to predict an outcome with respect to the condition (e.g., presence, indolence, recurrence, or progression). For example, an analytical tool may be used to generate a predictive model based on data for a cohort of patients whose outcomes with respect to a medical condition (e.g., time to recurrence or progression of cancer) are at least partially known. The model may then be used to evaluate data for a new patient in order to predict the risk of occurrence of the medical condition in the new patient. In some embodiments, only a subset of clinical, molecular, morphometric, and/or other data (e.g., clinical and morphometric data only) may be used by the analytical tool to generate the predictive model. Illustrative systems and methods for treating, diagnosing, and predicting the occurrence of medical conditions are described in commonly-owned U.S. Pat. No. 7,461,048, issued Dec. 2, 2008, U.S. Pat. No. 7,467,119, issued Dec. 16, 2008, PCT Application No. PCT/US2008/004523, filed Apr. 7, 2008, U.S. Publication No. 20100177950, published Jul. 15, 2010, and U.S. Publication No. 20100184093, published Jul. 22, 2010, which are all hereby incorporated by reference herein in their entireties.

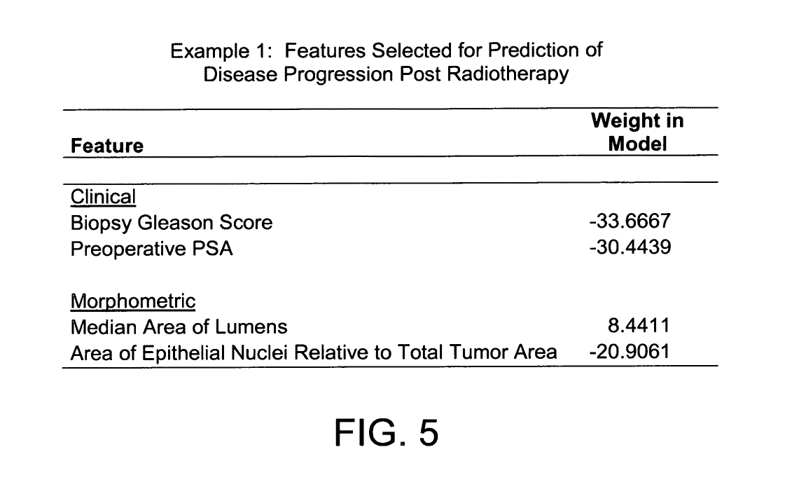

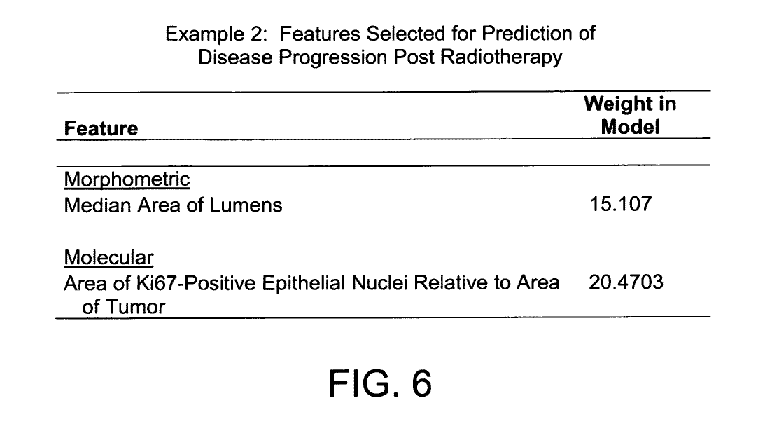

The clinical, molecular, and/or morphometric data used by embodiments of the present invention may include any clinical, molecular, and/or morphometric data that is relevant to the diagnosis, treatment and/or prediction of a medical condition. For example, features analyzed for correlations with progression of prostate cancer in a patient even after radiation therapy are described below in connection with FIGS. 5 and 6 and Tables 2, 3, and 4. It will be understood that at least some of these features may provide a basis for developing predictive models for other medical conditions (e.g., breast, colon, lung, bladder, liver, pancreas, renal cell, and soft tissue). For example, one or more of these features may be assessed for patients having some other medical condition and then input to an analytical tool that determines whether the features correlate with the medical condition. Generally, features that increase the ability of the model to predict the occurrence of the medical condition (e.g., as determined through suitable univariate and/or multivariate analyses) may be included in the final model, whereas features that do not increase (e.g., or decrease) the predictive power of the model may be removed from consideration. By way of example only, illustrative systems and methods for selecting features for use in a predictive model are described below and in commonly-owned U.S. Publication No. 2007/0112716, published May 17, 2007 and entitled “Methods and Systems for Feature Selection in Machine Learning Based on Feature Contribution and Model Fitness,” which is hereby incorporated by reference herein in its entirety.

Using the features in FIGS. 5 and 6 and Tables 2, 3, and 4 as a basis for developing a predictive model may focus the resources of physicians, other individuals, and/or automated processing equipment (e.g., a tissue image analysis system) on obtaining patient data that is more likely to be correlated with outcome and therefore useful in the final predictive model. Moreover, the features determined to be correlated with progression of prostate cancer in a patient even after radiation therapy are shown in FIGS. 5 and 6 and Tables 2, 3, and 4. It will be understood that these features may be included directly in final models predictive of such progression of prostate cancer, respectively, and/or used for developing predictive models for other medical conditions.

The morphometric data used in predictive models according to some embodiments of the present invention may include computer-generated data indicating various structural, textural, and/or spectral properties of, for example, tissue specimens. For example, the morphometric data may include data for morphometric features of stroma, cytoplasm, epithelial nuclei, stroma nuclei, lumen, red blood cells, tissue artifacts, tissue background, glands, other objects identified in a tissue specimen or a digitized image of such tissue, or a combination thereof.

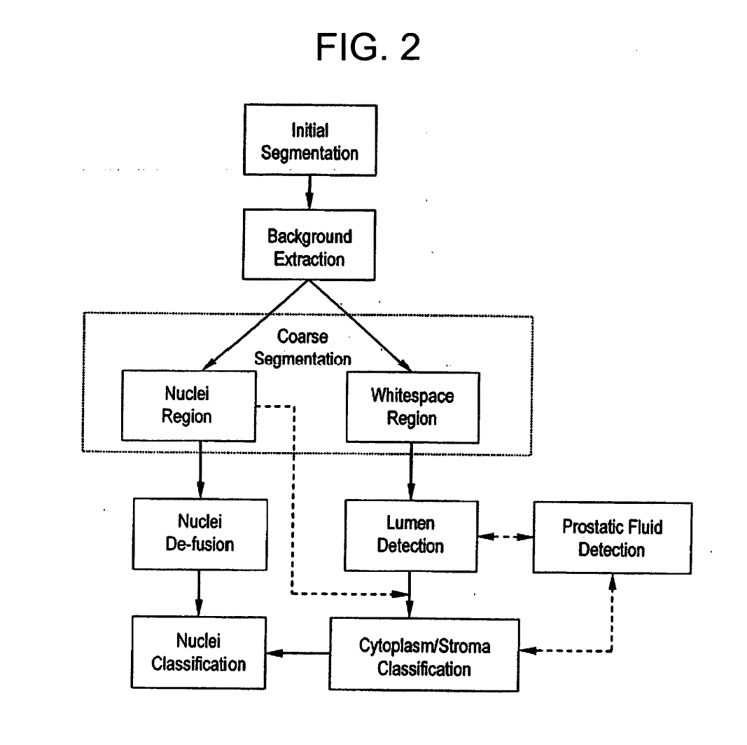

In an aspect of the present invention, a tissue image analysis system is provided for measuring morphometric features from tissue specimen(s) (e.g., needle biopsies and/or whole tissue cores) or digitized image(s) thereof. The system may utilize, in part, the commercially-available Definiens Cellenger software. For example, in some embodiments, the image analysis system may receive image(s) of tissue stained with hematoxylin and eosin (H&E) as input, and may output one or more measurements of morphometric features for pathological objects (e.g., epithelial nuclei, cytoplasm, etc.) and/or structural, textural, and/or spectral properties observed in the image(s). For example, such an image analysis system may include a light microscope that captures images of H&E-stained tissue at 20× magnification and/or at 40× magnification. Illustrative systems and methods for measuring morphometric features from images of H&E-stained tissue according to some embodiments of the present invention are described below in connection with, for example, FIG. 2 and the illustrative studies in which aspects of the present invention were applied to prediction of progression of prostate cancer in a patient even after radiation therapy. Computer-generated morphometric features (e.g., morphometric features measurable from digitized images of H&E-stained tissue) which may be used in a predictive model for predicting an outcome with respect to a medical condition according to some embodiments of the present invention are summarized in Table 1 of above-incorporated, commonly-owned U.S. Publication No. 20100184093.

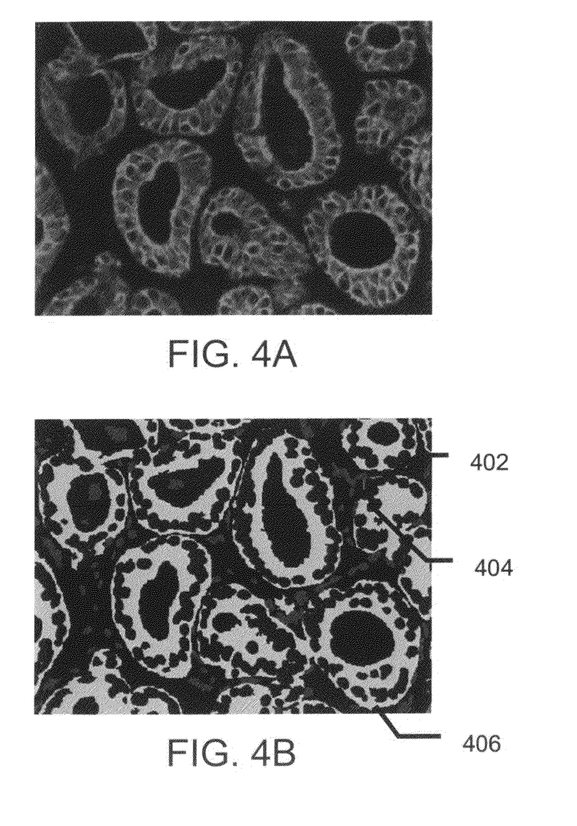

In some embodiments of the present invention, the image analysis system may receive image(s) of tissue subject to multiplex immunofluorescence (IF) as input, and may output one or more measurements of morphometric features for pathological objects (e.g., epithelial nuclei, cytoplasm, etc.) and/or structural, textural, and/or spectral properties observed in the image(s). For example, such an image analysis system may include a multispectral camera attached to a microscope that captures images of tissue under an excitation light source. Computer-generated morphometric features (e.g., morphometric features measurable from digitized images of tissue subject to multiplex IF) which may be used in a predictive model for predicting an outcome with respect to a medical condition according to some embodiments of the present invention are listed in Table 2 of above-incorporated, commonly-owned U.S. Publication No. 20100184093. Illustrative examples of such morphometric features include characteristics of a minimum spanning tree (MST) (e.g., MST connecting epithelial nuclei) and/or a fractal dimension (FD) (e.g., FD of gland boundaries) measured in images acquired through multiplex IF microscopy. Additional details regarding illustrative systems and methods for measuring morphometric features from images of tissue subject to multiplex IF according to some embodiments of the present invention are described in above-incorporated, commonly-owned U.S. Publication No. 20100184093 in connection with, for example, FIGS. 4B-9.

Clinical features which may be used in predictive models according to some embodiments of the present invention may include or be based on data for one or more patients such as age, race, weight, height, medical history, genotype and disease state, where disease state refers to clinical and pathologic staging characteristics and any other clinical features gathered specifically for the disease process under consideration. Generally, clinical data is gathered by a physician during the course of examining a patient and/or the tissue or cells of the patient. The clinical data may also include clinical data that may be more specific to a particular medical context. For example, in the context of prostate cancer, the clinical data may include data indicating blood concentration of prostate specific antigen (PSA), the result of a digital rectal exam, Gleason score, and/or other clinical data that may be more specific to prostate cancer. Clinical features which may be used in a predictive model for predicting an outcome with respect to a medical condition according to some embodiments of the present invention are listed in Table 4 of above-incorporated, commonly-owned U.S. Publication No. 20100184093.

Molecular features which may be used in predictive models according to some embodiments of the present invention may include or be based on data indicating the presence, absence, relative increase or decrease or relative location of biological molecules including nucleic acids, polypeptides, saccharides, steroids and other small molecules or combinations of the above, for example, glycoproteins and protein-RNA complexes. The locations at which these molecules are measured may include glands, tumors, stroma, and/or other locations, and may depend on the particular medical context. Generally, molecular data is gathered using molecular biological and biochemical techniques including Southern, Western, and Northern blots, polymerase chain reaction (PCR), immunohistochemistry, and/or immunofluorescence (IF) (e.g., multiplex IF). Molecular features which may be used in a predictive model for predicting an outcome with respect to a medical condition according to some embodiments of the present invention are listed in Table 3 of above-incorporated, commonly-owned U.S. Publication No. 20100184093. Additional details regarding multiplex immunofluorescence according to some embodiments of the present invention are described in commonly-owned U.S. Patent Application Publication No. 2007/0154958, published Jul. 5, 2007 and entitled “Multiplex In Situ Immunohistochemical Analysis,” which is hereby incorporated by reference herein in its entirety. Further, in situ hybridization may be used to show both the relative abundance and location of molecular biological features. Illustrative methods and systems for in situ hybridization of tissue are described in, for example, commonly-owned U.S. Pat. No. 6,995,020, issued Feb. 7, 2006 and entitled “Methods and compositions for the preparation and use of fixed-treated cell-lines and tissue in fluorescence in situ hybridization,” which is hereby incorporated by reference herein in its entirety.

Generally, when any clinical, molecular, and/or morphometric features from any of FIGS. 5 and 6 and Tables 2, 3, and 4 of the present disclosure, or Tables 1-4 of above-incorporated, commonly-owned U.S. Publication No. 20100184093, are applied to medical contexts other than the prostate, features from these Tables and/or Figures that are more specific to the prostate may not be considered. Optionally, features more specific to the medical context in question may be substituted for the prostate-specific features. For example, other histologic disease-specific features/manifestations may include regions of necrosis (e.g., ductal carcinoma in situ for the breast), size, shape and regional pattern/distribution of epithelial cells (e.g., breast, lung), degree of differentiation (e.g., squamous differentiation with non-small cell lung cancer (NSCLC, mucin production as seen with various adenocarcinomas seen in both breast and colon)), morphological/microscopic distribution of the cells (e.g., lining ducts in breast cancer, lining bronchioles in NSCLC), and degree and type of inflammation (e.g., having different characteristics for breast and NSCLC in comparison to prostate).

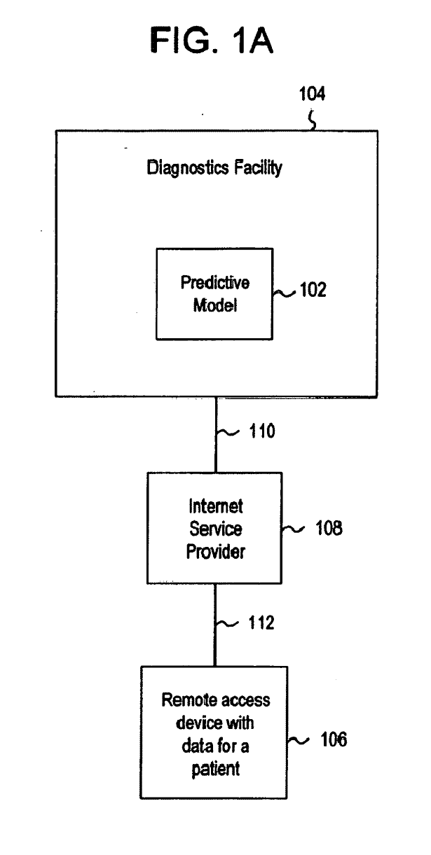

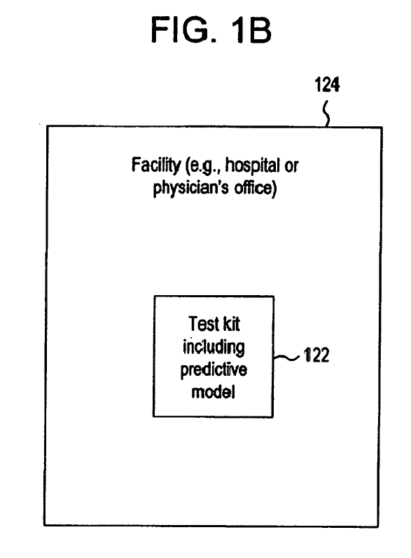

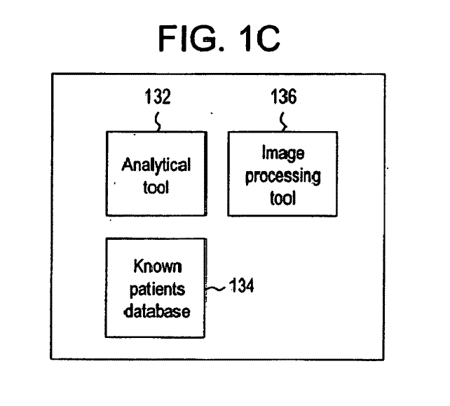

FIGS. 1A and 1B show illustrative systems that use a predictive model to predict the occurrence (e.g., presence, indolence, recurrence, or progression) of a medical condition in a patient. The arrangement in FIG. 1A may be used when, for example, a medical diagnostics lab provides support for a medical decision to a physician or other individual associated with a remote access device. The arrangement in FIG. 1B may be used when, for example, a test kit including the predictive model according to some embodiments of the present invention is provided for use in a facility such as a hospital, other medical facility, or other suitable location.

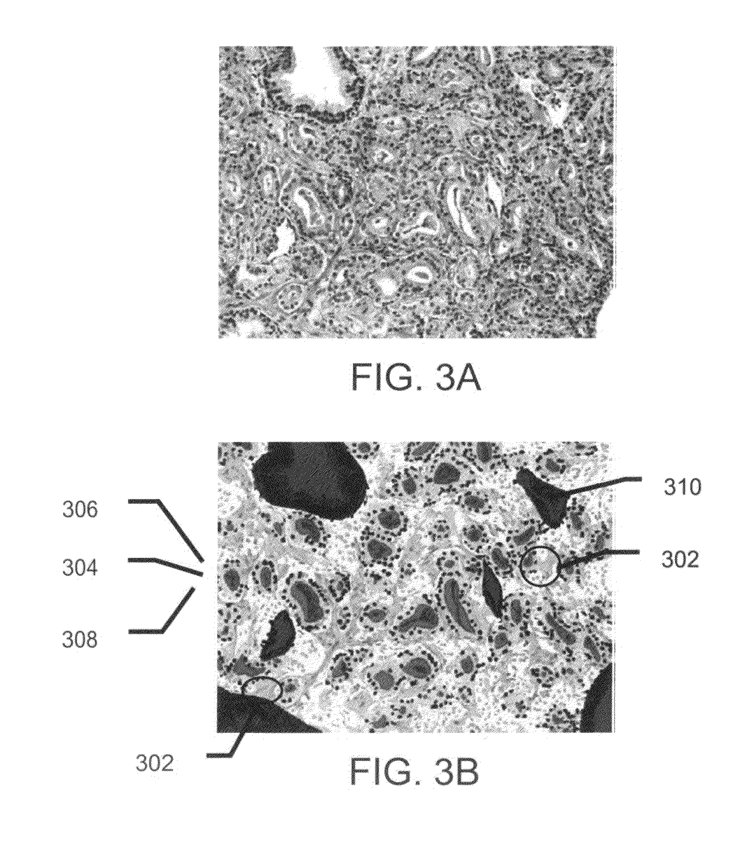

Referring to FIG. 1A, one or more predictive models 102 are located in diagnostics facility 104. Predictive model(s) 102 may include any suitable hardware, software, or combination thereof for receiving data for a patient, evaluating the data in order to predict the occurrence (e.g., presence, indolence, recurrence, and/or progression) of a medical condition for the patient, and outputting the results of the evaluation. In another embodiment, a model 102 may be used to predict the responsiveness of a patient to particular one or more therapies. Diagnostics facility 104 may receive data for a patient from remote access device 106 via Internet service provider (ISP) 108 and communications networks 110 and 112, and may input the data to predictive model(s) 102 for evaluation. Other arrangements for receiving and evaluating data for a patient from a remote location are of course possible (e.g., via another connection such as a telephone line or through the physical mail). The remotely located physician or individual may acquire the data for the patient in any suitable manner and may use remote access device 106 to transmit the data to diagnostics facility 104. In some embodiments, the data for the patient may be at least partially generated by diagnostics facility 104 or another facility. For example, diagnostics facility 104 may receive a digitized image of H&E-stained tissue from remote access device 106 or other device and may generate morphometric data for the patient based on the image. In another example, actual tissue samples may be received and processed by diagnostics facility 104 in order to generate morphometric data, molecular data, and/or other data. In other examples, a third party may receive a tissue sample or image for a new patient, generate morphometric data, molecular data and/or other data based on the image or tissue, and provide the morphometric data, molecular data and/or other data to diagnostics facility 104. Additional details regarding illustrative embodiments of suitable image processing tools for generating morphometric data and/or molecular data from tissue images and/or tissue samples according to some embodiments of the present invention are described in connection with FIGS. 3-8 of above-incorporated, commonly-owned U.S. Publication No. 20100184093.

Diagnostics facility 104 may provide the results of the evaluation to a physician or individual associated with remote access device 106 through, for example, a transmission to remote access device 106 via ISP 108 and communications networks 110 and 112 or in another manner such as the physical mail or a telephone call. The results may include one or more values or “scores” (e.g., an indication of the likelihood that the patient will experience one or more outcomes related to the medical condition such as the presence of the medical condition, or risk or likelihood of progression of the medical condition in the patient even after radiotherapy), information indicating one or more features analyzed by predictive model(s) 102 as being correlated with the medical condition, image(s) output by the image processing tool, information indicating the sensitivity and/or specificity of the predictive model, explanatory remarks, other suitable information, or a combination thereof. In some embodiments, the information may be provided in a report that may be used by a physician or other individual, for example, to assist in determining appropriate treatment option(s) for the patient. The report may also be useful in that it may help the physician or individual to explain the patient's risk to the patient.

Remote access device 106 may be any remote device capable of transmitting and/or receiving data from diagnostics facility 104 such as, for example, a personal computer, a wireless device such as a laptop computer, a cell phone or a personal digital assistant (PDA), or any other suitable remote access device. Multiple remote access devices 106 may be included in the system of FIG. 1A (e.g., to allow a plurality of physicians or other individuals at a corresponding plurality of remote locations to communicate data with diagnostics facility 104), although only one remote access device 106 has been included in FIG. 1A to avoid over-complicating the drawing. Diagnostics facility 104 may include a server capable of receiving and processing communications to and/or from remote access device 106. Such a server may include a distinct component of computing hardware and/or storage, but may also be a software application or a combination of hardware and software. The server may be implemented using one or more computers.

Each of communications links 110 and 112 may be any suitable wired or wireless communications path or combination of paths such as, for example, a local area network, wide area network, telephone network, cable television network, intranet, or Internet. Some suitable wireless communications networks may be a global system for mobile communications (GSM) network, a time-division multiple access (TDMA) network, a code-division multiple access (CDMA) network, a Bluetooth network, or any other suitable wireless network.

FIG. 1B shows a system in which test kit 122 including a predictive model in accordance with an embodiment of the present invention is provided for use in facility 124, which may be a hospital, a physician's office, or other suitable location. Test kit 122 may include any suitable hardware, software, or combination thereof (e.g., a personal computer) that is adapted to receive data for a patient (e.g., at least one of clinical, morphometric and molecular data), evaluate the patient's data with one or more predictive models (e.g., programmed in memory or other non-transitory computer readable media of the test kit), and output the results of the evaluation. For example, test kit 122 may include a computer readable medium encoded with computer executable instructions for performing the functions of the predictive model(s). The predictive model(s) may be predetermined model(s) previously generated (e.g., by another system or application such as the system in FIG. 1C). In some embodiments, test kit 122 may optionally include an image processing tool capable of generating data corresponding to morphometric and/or molecular features from, for example, a tissue sample or image. In other embodiments, test kit 122 may receive pre-packaged data for the morphometric features as input from, for example, an input device (e.g., keyboard) or another device or location. Test kit 122 may optionally include an input for receiving, for example, updates to the predictive model. The test kit may also optionally include an output for transmitting data, such as data useful for patient billing and/or tracking of usage, to a main facility or other suitable device or location. The billing data may include, for example, medical insurance information for a patient evaluated by the test kit (e.g., name, insurance provider, and account number). Such information may be useful when, for example, a provider of the test kit charges for the kit on a per-use basis and/or when the provider needs patients' insurance information to submit claims to insurance providers.

FIG. 1C shows an illustrative system for generating a predictive model according to some embodiments of the present invention. The system includes analytical tool 132 (e.g., including a module configured to perform support vector regression for censored data (SVRc), a support vector machine (SVM), and/or a neural network) and database 134 of patients whose outcomes are at least partially known. Analytical tool 132 may include any suitable hardware, software, or combination thereof for determining correlations between the data from database 134 and a medical condition. The system in FIG. 1C may also include image processing tool 136 capable of generating, for example, morphometric data based on H&E-stained tissue or digitized image(s) thereof, morphometric data and/or molecular data based on tissue acquired using multiplex immunofluorescence (IF) microscopy or digitized image(s) of such tissue, or a combination thereof. Tool 136 may generate morphometric data and/or molecular data for, for example, the known patients whose data is included in database 134.

Database 134 may include any suitable patient data such as data for clinical features, morphometric features, molecular features, or a combination thereof. Database 134 may also include data indicating the outcomes of patients such as whether and when the patients have experienced a disease or its recurrence or progression. For example, database 134 may include uncensored data for patients (i.e., data for patients whose outcomes are completely known) such as data for patients who have experienced a medical condition (e.g., favorable or unfavorable pathological stage) or its recurrence or progression. Database 134 may alternatively or additionally include censored data for patients (i.e., data for patients whose outcomes are not completely known) such as data for patients who have not shown signs of a disease or its recurrence or progression in one or more follow-up visits to a physician (e.g., follow-up visits post radiotherapy). The use of censored data by analytical tool 132 may increase the amount of data available to generate the predictive model and, therefore, may advantageously improve the reliability and predictive power of the model. Examples of machine learning approaches, namely support vector regression for censored data (SVRc) and a particular implementation of a neural network (NNci) that can make use of both censored and uncensored data are described below.

In one embodiment, analytical tool 132 may perform support vector regression on censored data (SVRc) in the manner set forth in commonly-owned U.S. Pat. No. 7,505,948, issued Mar. 17, 2009, which is hereby incorporated by reference herein in its entirety. SVRc uses a loss/penalty function which is modified relative to support vector machines (SVM) in order to allow for the utilization of censored data. For example, data including clinical, molecular, and/or morphometric features of known patients from database 134 may be input to the SVRc to determine parameters for a predictive model. The parameters may indicate the relative importance of input features, and may be adjusted in order to maximize the ability of the SVRc to predict the outcomes of the known patients.

The use of SVRc by analytical tool 132 may include obtaining from database 134 multi-dimensional, non-linear vectors of information indicative of status of patients, where at least one of the vectors lacks an indication of a time of occurrence of an event or outcome with respect to a corresponding patient. Analytical tool 132 may then perform regression using the vectors to produce a kernel-based model that provides an output value related to a prediction of time to the event based upon at least some of the information contained in the vectors of information. Analytical tool 132 may use a loss function for each vector containing censored data that is different from a loss function used by tool 132 for vectors comprising uncensored data. A censored data sample may be handled differently because it may provide only “one-sided information.” For example, in the case of survival time prediction, a censored data sample typically only indicates that the event has not happened within a given time, and there is no indication of when it will happen after the given time, if at all.

The loss function used by analytical tool 132 for censored data may be as follows:

$$Loss ( f ( x ) , y , s = 1 ) = \{ \begin{matrix}{C}_{s}^{*} ( e - {ɛ}_{s}^{*} ) & e > {ɛ}_{s}^{*} \\ 0 & - {ɛ}_{s} \leq e \leq {ɛ}_{s}^{*} \\ {C}_{s} ( {ɛ}_{s} - e ) & e < - {ɛ}_{s} ,\end{matrix}$$

where e=f(x)−y; and

f(x)=W^{T}Φ(x)+b

is a linear regression function on a feature space F. Here, W is a vector in F, and Φ(x) maps the input x to a vector in F.

In contrast, the loss function used by tool 132 for uncensored data may be:

$$Loss ( f ( x ) , y , s = 0 ) = \{ \begin{matrix}{C}_{n}^{*} ( e - {ɛ}_{n}^{*} ) & e > {ɛ}_{n}^{*} \\ 0 & - {ɛ}_{n} \leq e \leq {ɛ}_{n}^{*} \\ {C}_{n} ( {ɛ}_{n} - e ) & e < - {ɛ}_{n} ,\end{matrix}$$

where e=f(x)−y

and ε*ₙ≦εₙ and C*ₙ≧Cₙ.

In the above description, the W and b are obtained by solving an optimization problem, the general form of which is:

$$\begin{matrix}min \\ W , b\end{matrix} \frac{1}{2} {W}^{T} W$$

$$s . t . {y}_{i} - ( {W}^{T} φ ( {x}_{i} ) + b ) \leq ɛ \\ ( {W}^{T} φ ( {x}_{i} ) + b ) - {y}_{i} \leq ɛ$$

This equation, however, assumes the convex optimization problem is always feasible, which may not be the case. Furthermore, it is desired to allow for small errors in the regression estimation. It is for these reasons that a loss function is used for SVRc. The loss allows some leeway for the regression estimation. Ideally, the model built will exactly compute all results accurately, which is infeasible. The loss function allows for a range of error from the ideal, with this range being controlled by slack variables ξ and ξ*, and a penalty C. Errors that deviate from the ideal, but are within the range defined by ξ and ξ*, are counted, but their contribution is mitigated by C. The more erroneous the instance, the greater the penalty. The less erroneous (closer to the ideal) the instance is, the less the penalty. This concept of increasing penalty with error results in a slope, and C controls this slope. While various loss functions may be used, for an epsilon-insensitive loss function, the general equation transforms into:

$${min}_{W , b} P = \frac{1}{2} {W}^{T} W + C {\sum }_{i = 1}^{l} ( {ξ}_{i} + {ξ}_{i}^{*} )$$

$$s . t . {y}_{i} - ( {W}^{T} Φ ( {x}_{i} ) + b ) \leq ɛ + {ξ}_{i} \\ ( {W}^{T} Φ ( {x}_{i} ) + b ) - {y}_{i} \leq ɛ + {ξ}_{i}^{*}$$

$${ξ}_{i} , {ξ}_{i}^{*} \geq 0 , i = 1 … l$$

For an epsilon-insensitive loss function in accordance with the invention (with different loss functions applied to censored and uncensored data), this equation becomes:

$${min}_{W , b} {P}_{c} = \frac{1}{2} {W}^{T} W + {\sum }_{i = 1}^{l} ( {C}_{i} {ξ}_{i} + {C}_{i}^{*} {ξ}_{i}^{*} )$$

$$s . t . {y}_{i} - ( {W}^{T} Φ ( {x}_{i} ) + b ) \leq {ɛ}_{i} + {ξ}_{i} \\ ( {W}^{T} Φ ( {x}_{i} ) + b ) - {y}_{i} \leq {ɛ}_{i}^{*} + {ξ}_{i}^{*}$$

$${ξ}_{i}^{(* )} \geq 0 , i = 1 … l$$

$$where {C}_{i}^{(* )} = {s}_{i} {C}_{s}^{(* )} + ( 1 - {s}_{i} ) {C}_{n}^{(* )}$$

$${ɛ}_{i}^{(* )} = {s}_{i} {ɛ}_{s}^{(* )} + ( 1 - {s}_{i} ) {ɛ}_{n}^{(* )}$$

The optimization criterion penalizes data points whose y-values differ from f(x) by more than ε. The slack variables, ξ and ξ*, correspond to the size of this excess deviation for positive and negative deviations respectively. This penalty mechanism has two components, one for uncensored data (i.e., not right-censored) and one for censored data. Here, both components are represented in the form of loss functions that are referred to as ε-insensitive loss functions.

In another embodiment, analytical tool 132 may include a module configured to perform binary logistic regression utilizing, at least in part, a commercially-available SAS computer package configured for regression analyses.

In yet another embodiment, analytical tool 132 may include a neural network. In such an embodiment, tool 132 preferably includes a neural network that is capable of utilizing censored data. Additionally, the neural network preferably uses an objective function substantially in accordance with an approximation (e.g., derivative) of the concordance index (CI) to train an associated model (NNci). Though the CI has long been used as a performance indicator for survival analysis, the use of the CI to train a neural network was proposed in commonly-owned U.S. Pat. No. 7,321,881, issued Jan. 22, 2008, which is hereby incorporated by reference herein in its entirety. The difficulty of using the CI as a training objective function in the past is that the CI is non-differentiable and cannot be optimized by gradient-based methods. As described in above-incorporated U.S. Pat. No. 7,321,881, this obstacle may be overcome by using an approximation of the CI as the objective function.

For example, when analytical tool 132 includes a neural network that is used to predict prostate cancer progression, the neural network may process input data for a cohort of patients whose outcomes with respect to prostate cancer progression are at least partially known in order to produce an output. The particular features selected for input to the neural network may be selected through the use of the above-described SVRc (e.g., implemented with analytical tool 132) or any other suitable feature selection process. An error module of tool 132 may determine an error between the output and a desired output corresponding to the input data (e.g., the difference between a predicted outcome and the known outcome for a patient). Analytical tool 132 may then use an objective function substantially in accordance with an approximation of the CI to rate the performance of the neural network. Analytical tool 132 may adapt the weighted connections (e.g., relative importance of features) of the neural network based upon the results of the objective function.

The concordance index may be expressed in the form:

$$CI = \frac{{\sum }_{( i , j ) ∈ Ω} I ( {\hat{t}}_{i} , {\hat{t}}_{j} )}{\vert Ω \vert}$$

$$where$$

$$I ( {\hat{t}}_{i} , {\hat{t}}_{j} ) = \{ \begin{matrix}1 \text{:} {\hat{t}}_{i} > {\hat{t}}_{j} \\ 0 \text{:} otherwise\end{matrix} \} ,$$

and may be based on pair-wise comparisons between the prognostic estimates \hat{t}ᵢ and \hat{t}ⱼ for patients i and j, respectively. In this example, Ω consists of all the pairs of patients {i,j} who meet the following conditions:
both patients i and j experienced recurrence, and the recurrence time tᵢ of patient i is shorter than patient j's recurrence time tⱼ; or
only patient i experienced recurrence and tᵢ is shorter than patient j's follow-up visit time tⱼ.
   The numerator of the CI represents the number of times that the patient predicted to recur earlier by the neural network actually does recur earlier. The denominator is the total number of pairs of patients who meet the predetermined conditions.

Generally, when the CI is increased, preferably maximized, the model is more accurate. Thus, by preferably substantially maximizing the CI, or an approximation of the CI, the performance of a model is improved. In accordance with some embodiments of the present invention, an approximation of the CI is provided as follows:

$$C = \frac{{\sum }_{( i , j ) ∈ Ω} R ( {\hat{t}}_{i} , {\hat{t}}_{j} )}{\vert Ω \vert}$$

$$where$$

$$R ( {\hat{t}}_{i} , {\hat{t}}_{j} ) = \{ \begin{matrix}{( - ( {\hat{t}}_{i} - {\hat{t}}_{j} - γ ) )}^{n} \text{:} {\hat{t}}_{i} - {\hat{t}}_{j} < γ \\ 0 \text{:} otherwise\end{matrix} \} ,$$

and where 0<γ≦1 and n>1. R(\hat{t}ᵢ,\hat{t}ⱼ) can be regarded as an approximation to I(−\hat{t}ᵢ,−\hat{t}ⱼ).

Another approximation of the CI provided in accordance with some embodiments of the present invention which has been shown empirically to achieve improved results is the following:

$${C}_{ω} = \frac{{\sum }_{( i , j ) ∈ Ω} - ( {\hat{t}}_{i} - {\hat{t}}_{j} ) \cdot R ( {\hat{t}}_{i} , {\hat{t}}_{j} )}{D} , \\ where$$

$$D = {\sum }_{( i , j ) ∈ Ω} - ( {\hat{t}}_{i} - {\hat{t}}_{j} )$$

is a normalization factor. Here each R(\hat{t}ᵢ,\hat{t}ⱼ) is weighted by the difference between \hat{t}ᵢ and \hat{t}ⱼ. The process of minimizing the C_{ω}, (or C) seeks to move each pair of samples in Ω to satisfy \hat{t}ᵢ−\hat{t}ⱼ>γ and thus to make I(\hat{t}ᵢ,\hat{t}ⱼ)=1.

When the difference between the outputs of a pair in Ω is larger than the margin γ, this pair of samples will stop contributing to the objective function. This mechanism effectively overcomes over-fitting of the data during training of the model and makes the optimization preferably focus on only moving more pairs of samples in Ω to satisfy \hat{t}ᵢ−\hat{t}ⱼ≧γ. The influence of the training samples is adaptively adjusted according to the pair-wise comparisons during training. Note that the positive margin γ in R is preferable for improved generalization performance. In other words, the parameters of the neural network are adjusted during training by calculating the CI after all the patient data has been entered. The neural network then adjusts the parameters with the goal of minimizing the objective function and thus maximizing the CI. As used above, over-fitting generally refers to the complexity of the neural network. Specifically, if the network is too complex, the network will react to “noisy” data. Overfitting is risky in that it can easily lead to predictions that are far beyond the range of the training data.

Morphometric Data Obtained from H&E-Stained Tissue

As described above, an image processing tool (e.g., image processing tool 136) in accordance with some embodiments of the present invention may be provided that generates digitized images of tissue specimens (e.g., H&E-stained tissue specimens) and/or measures morphometric features from the tissue images or specimens. For example, in some embodiments, the image processing tool may include a light microscope that captures tissue images (e.g., at 20× and/or 40× magnification) using a SPOT Insight QE Color Digital Camera (KAI2000) and produces images with 1600×1200 pixels. The images may be stored as images with 24 bits per pixel in Tiff format. Such equipment is only illustrative and any other suitable image capturing equipment may be used without departing from the scope of the present invention.

In some embodiments, the image processing tool may include any suitable hardware, software, or combination thereof for segmenting and classifying objects in the captured images, and then measuring morphometric features of the objects. For example, such segmentation of tissue images may be utilized in order to classify pathological objects in the images (e.g., classifying objects as cytoplasm, lumen, nuclei, epithelial nuclei, stroma, background, artifacts, red blood cells, glands, other object(s) or any combination thereof). In one embodiment, the image processing tool may include the commercially-available Definiens Cellenger Developer Studio (e.g., v. 4.0) adapted to perform the segmenting and classifying of, for example, some or all of the various pathological objects described above and to measure various morphometric features of these objects. Additional details regarding the Definiens Cellenger product are described in Definiens Cellenger Architecture: A Technical Review, April 2004, which is hereby incorporated by reference herein in its entirety.

For example, in some embodiments of the present invention, the image processing tool may classify objects as background if the objects correspond to portions of the digital image that are not occupied by tissue. Objects classified as cytoplasm may be the cytoplasm of a cell, which may be an amorphous area (e.g., pink area that surrounds an epithelial nucleus in an image of, for example, H&E stained tissue). Objects classified as epithelial nuclei may be the nuclei present within epithelial cells/luminal and basal cells of the glandular unit, which may appear as round objects surrounded by cytoplasm. Objects classified as lumen may be the central glandular space where secretions are deposited by epithelial cells, which may appear as enclosed white areas surrounded by epithelial cells. Occasionally, the lumen can be filled by prostatic fluid (which typically appears pink in H&E stained tissue) or other “debris” (e.g., macrophages, dead cells, etc.). Together the lumen and the epithelial cytoplasm and nuclei may be classified as a gland unit. Objects classified as stroma may be the connective tissue with different densities that maintains the architecture of the prostatic tissue. Such stroma tissue may be present between the gland units, and may appear as red to pink in H&E stained tissue. Objects classified as stroma nuclei may be elongated cells with no or minimal amounts of cytoplasm (fibroblasts). This category may also include endothelial cells and inflammatory cells, and epithelial nuclei may also be found scattered within the stroma if cancer is present. Objects classified as red blood cells may be small red round objects usually located within the vessels (arteries or veins), but can also be found dispersed throughout tissue.

In some embodiments, the image processing tool may measure various morphometric features of from basic relevant objects such as epithelial nuclei, epithelial cytoplasm, stroma, and lumen (including mathematical descriptors such as standard deviations, medians, and means of objects), spectral-based characteristics (e.g., red, green, blue (RGB) channel characteristics such as mean values, standard deviations, etc.), texture, wavelet transform, fractal code and/or dimension features, other features representative of structure, position, size, perimeter, shape (e.g., asymmetry, compactness, elliptic fit, etc.), spatial and intensity relationships to neighboring objects (e.g., contrast), and/or data extracted from one or more complex objects generated using said basic relevant objects as building blocks with rules defining acceptable neighbor relations (e.g., ‘gland unit’ features). In some embodiments, the image processing tool may measure these features for every instance of every identified pathological object in the image, or a subset of such instances. The image processing tool may output these features for, for example, evaluation by predictive model 102 (FIG. 1A), test kit 122 (FIG. 1B), or analytical tool 132 (FIG. 1C). Optionally, the image processing tool may also output an overall statistical summary for the image summarizing each of the measured features.

FIG. 2 is a flowchart of illustrative stages involved in image segmentation and object classification (e.g., in digitized images of H&E-stained tissue) according to some embodiments of the present invention.

Initial Segmentation. In a first stage, the image processing tool may segment an image (e.g., an H&E-stained needle biopsy tissue specimen, an H&E stained tissue microarray (TMA) image or an H&E of a whole tissue section) into small groups of contiguous pixels known as objects. These objects may be obtained by a region-growing method which finds contiguous regions based on color similarity and shape regularity. The size of the objects can be varied by adjusting a few parameters, as described in Baatz M. and Schäpe A., “Multiresolution Segmentation—An Optimization Approach for High Quality Multi-scale Image Segmentation,” In Angewandte Geographische Informationsverarbeitung XII, Strobl, J., Blaschke, T., Griesebner, G. (eds.), Wichmann-Verlag, Heidelberg, 12-23, 2000, which is hereby incorporated by reference herein in its entirety. In this system, an object rather than a pixel is typically the smallest unit of processing. Thus, some or all of the morphometric feature calculations and operations may be performed with respect to objects. For example, when a threshold is applied to the image, the feature values of the object are subject to the threshold. As a result, all the pixels within an object are assigned to the same class. In one embodiment, the size of objects may be controlled to be 10-20 pixels at the finest level. Based on this level, subsequent higher and coarser levels are built by forming larger objects from the smaller ones in the lower level.

Background Extraction. Subsequent to initial segmentation, the image processing tool may segment the image tissue core from the background (transparent region of the slide) using intensity threshold and convex hull. The intensity threshold is an intensity value that separates image pixels in two classes: “tissue core” and “background.” Any pixel with an intensity value greater than or equal the threshold is classified as a “tissue core” pixel, otherwise the pixel is classified as a “background” pixel. The convex hull of a geometric object is the smallest convex set (polygon) containing that object. A set S is convex if, whenever two points P and Q are inside S, then the whole line segment PQ is also in S.

Coarse Segmentation. In a next stage, the image processing tool may re-segment the foreground (e.g., TMA core) into rough regions corresponding to nuclei and white spaces. For example, the main characterizing feature of nuclei in H&E stained images is that they are stained blue compared to the rest of the pathological objects. Therefore, the difference in the red and blue channels (R−B) intensity values may be used as a distinguishing feature. Particularly, for every image object obtained in the initial segmentation step, the difference between average red and blue pixel intensity values may be determined. The length/width ratio may also be used to determine whether an object should be classified as nuclei area. For example, objects which fall below a (R−B) feature threshold and below a length/width threshold may be classified as nuclei area. Similarly, a green channel threshold can be used to classify objects in the tissue core as white spaces. Tissue stroma is dominated by the color red. The intensity difference d, “red ratio” r=R/(R+G+B) and the red channel standard deviation σ_{R} of image objects may be used to classify stroma objects.

White Space Classification. In the stage of coarse segmentation, the white space regions may correspond to both lumen (pathological object) and artifacts (broken tissue areas) in the image. The smaller white space objects (area less than 100 pixels) are usually artifacts. Thus, the image processing tool may apply an area filter to classify them as artifacts.

Nuclei De-fusion and Classification. In the stage of coarse segmentation, the nuclei area is often obtained as contiguous fused regions that encompass several real nuclei. Moreover, the nuclei region might also include surrounding misclassified cytoplasm. Thus, these fused nuclei areas may need to be de-fused in order to obtain individual nuclei.

The image processing tool may use two different approaches to de-fuse the nuclei. The first approach may be based on a region growing method that fuses the image objects constituting nuclei area under shape constraints (roundness). This approach has been determined to work well when the fusion is not severe.

In the case of severe fusion, the image processing tool may use a different approach based on supervised learning. This approach involves manual labeling of the nuclei areas by an expert (pathologist). The features of image objects belonging to the labeled nuclei may be used to design statistical classifiers.

In some embodiments, the input image may include different kinds of nuclei: epithelial nuclei, fibroblasts, basal nuclei, endothelial nuclei, apoptotic nuclei and red blood cells. Since the number of epithelial nuclei is typically regarded as an important feature in grading the extent of the tumor, it may be important to distinguish the epithelial nuclei from the others. The image processing tool may accomplish this by classifying the detected nuclei into two classes: epithelial nuclei and “the rest” based on shape (eccentricity) and size (area) features.

In one embodiment, in order to reduce the number of feature space dimensions, feature selection may be performed on the training set using two different classifiers: the Bayesian classifier and the k nearest neighbor classifier (F. E. Harrell et al., “Evaluating the yield of medical tests,” JAMA, 247(18):2543-2546, 1982, which is hereby incorporated by reference herein in its entirety). The leave-one-out method (Definiens Cellenger) may be used for cross-validation, and the sequential forward search method may be used to choose the best features. Finally, two Bayesian classifiers may be designed with number of features equal to 1 and 5, respectively. The class-conditional distributions may be assumed to be Gaussian with diagonal covariance matrices.

The image segmentation and object classification procedure described above in connection with FIG. 2 is only illustrative and any other suitable method or approach may be used to measure morphometric features of interest in tissue specimens or images in accordance with the present invention. For example, in some embodiments, a digital masking tool (e.g., Adobe Photoshop 7.0) may be used to mask portion(s) of the tissue image such that only infiltrating tumor is included in the segmentation, classification, and/or subsequent morphometric analysis. Alternatively or additionally, in some embodiments, lumens in the tissue images are manually identified and digitally masked (outlined) by a pathologist in an effort to minimize the effect of luminal content (e.g., crystals, mucin, and secretory concretions) on lumen object segmentation. Additionally, these outlined lumens can serve as an anchor for automated segmentation of other cellular and tissue components, for example, in the manner described below.

In some embodiments of the present invention, the segmentation and classification procedure identifies gland unit objects in a tissue image, where each gland unit object includes lumen, epithelial nuclei, and epithelial cytoplasm. The gland unit objects are identified by uniform and symmetric growth around lumens as seeds. Growth proceeds around these objects through spectrally uniform segmented epithelial cells until stroma cells, retraction artifacts, tissue boundaries, or other gland unit objects are encountered. These define the borders of the glands, where the accuracy of the border is determined by the accuracy of differentiating the cytoplasm from the remaining tissue. In this example, without addition of stop conditions, uncontrolled growth of connected glands may occur. Thus, in some embodiments, firstly the small lumens (e.g., very much smaller than the area of an average nucleus) are ignored as gland seeds. Secondly, the controlled region-growing method continues as long as the area of each successive growth ring is larger than the preceding ring. Segments of non-epithelial tissue are excluded from these ring area measurements and therefore effectively dampen and halt growth of asymmetric glands. The epithelial cells (including epithelial nuclei plus cytoplasm) thus not captured by the gland are classified as outside of, or poorly associated with, the gland unit. In this manner, epithelial cells (including epithelial nuclei plus cytoplasm) outside of the gland units are also identified.

In some embodiments, an image processing tool may be provided that classifies and clusters objects in tissue, which utilizes biologically defined constraints and high certainty seeds for object classification. In some embodiments, such a tool may rely less on color-based features than prior classification approaches. For example, a more structured approach starts with high certainty lumen seeds (e.g., based on expert outlined lumens) and using them as anchors, and distinctly colored object segmented objects. The distinction of lumens from other transparent objects, such as tissue tears, retraction artifacts, blood vessels and staining defects, provides solid anchors and object neighbor information to the color-based classification seeds. The probability distributions of the new seed object features, along with nearest neighbor and other clustering techniques, are used to further classify the remaining objects. Biological information regarding of the cell organelles (e.g., their dimensions, shape and location with respect to other organelles) constrains the growth of the classified objects. Due to tissue-to-tissue irregularities and feature outliers, multiple passes of the above approach may be used to label all the segments. The results are fed back to the process as new seeds, and the process is iteratively repeated until all objects are classified. In some embodiments, since at 20× magnification the nuclei and sub-nuclei objects may be too coarsely resolved to accurately measure morphologic features, measurements of nuclei shape, size and nuclei sub-structures (chromatin texture, and nucleoli) may be measured at 40× magnification (see e.g., Table 1 of above-incorporated, commonly-owned U.S. Publication No. 20100184093). To reduce the effect of segmentation errors, the 40× measurements may differentiate the feature properties of well defined nuclei (based on strongly defined boundaries of elliptic and circular shape) from other poorly differentiated nuclei.

FIG. 3A is an image of typical H&E-stained prostate tissue obtained via a needle biopsy. FIG. 3B is a segmented and classified version of the image in FIG. 3A according to some embodiments of the present invention, showing gland units 302 formed from seed lumen 304, epithelial nuclei 306, and epithelial cytoplasm 308. Also segmented and classified in the processed image are isolated/non-gland-associated tumor epithelial cells 310, which include epithelial nuclei and epithelial cytoplasm. Although in the original image the seed lumen 304, epithelial nuclei 306, and epithelial cytoplasm 308 of the gland units are red, dark blue, and light blue, respectively, and the epithelial nuclei and epithelial cytoplasm of the isolated/non-gland-associated tumor epithelial cells are green and clear, respectively, the image is provided in gray-scale in FIG. 3B for ease of reproducibility. Black/gray areas represent benign elements and tissue artifacts which have been digitally removed by the pathologist reviewing the case.

Additional details regarding image segmentation and measuring morphometric features of the classified pathological objects according to some embodiments of the present invention are described in above-incorporated U.S. Pat. No. 7,461,048, issued Dec. 2, 2008, U.S. Pat. No. 7,467,119, issued Dec. 16, 2008, PCT Application No. PCT/US2008/004523, filed Apr. 7, 2008, U.S. Publication No. 20100177950, published Jul. 15, 2010, and U.S. Publication No. 20100184093, published Jul. 22, 2010, as well as commonly-owned U.S. Publication No. 2006/0064248, published Mar. 23, 2006 and entitled “Systems and Methods for Automated Grading and Diagnosis of Tissue Images,” and U.S. Pat. No. 7,483,554, issued Jan. 27, 2009 and entitled “Pathological Tissue Mapping,” which are hereby incorporated by reference herein in their entireties.

Morphometric Data and/or Molecular Data Obtained from Multiplex IF

In some embodiments of the present invention, an image processing tool (e.g., image processing tool 136) is provided that generates digitized images of tissue specimens subject to immunofluorescence (IF) (e.g., multiplex IF) and/or measures morphometric and/or molecular features from the tissue images or specimens. In multiplex IF microscopy, multiple proteins in a tissue specimen are simultaneously labeled with different fluorescent dyes conjugated to antibodies specific for each particular protein. Each dye has a distinct emission spectrum and binds to its target protein within a tissue compartment such as nuclei or cytoplasm. Thus, the labeled tissue is imaged under an excitation light source using a multispectral camera attached to a microscope. The resulting multispectral image is then subjected to spectral unmixing to separate the overlapping spectra of the fluorescent labels. The unmixed multiplex IF images have multiple components, where each component represents the expression level of a protein in the tissue.

In some embodiments of the present invention, images of tissue subject to multiplex IF are acquired with a CRI Nuance spectral imaging system (CRI, Inc., 420-720 nm model) mounted on a Nikon 90i microscope equipped with a mercury light source (Nikon) and an Opti Quip 1600 LTS system. In some embodiments, DAPI nuclear counterstain is recorded at 480 nm wavelength using a bandpass DAPI filter (Chroma). Alexa 488 may be captured between 520 and 560 nm in 10 nm intervals using an FITC filter (Chroma). Alexa 555, 568 and 594 may be recorded between 570 and 670 nm in 10 nm intervals using a custom-made longpass filter (Chroma), while Alexa 647 may be recorded between 640 and 720 nm in 10 nm intervals using a second custom-made longpass filter (Chroma). Spectra of the pure dyes were recorded prior to the experiment by diluting each Alexa dye separately in SlowFade Antifade (Molecular Probes). In some embodiments, images are unmixed using the Nuance software Version 1.4.2, where the resulting images are saved as quantitative grayscale tiff images and submitted for analysis.

For example, FIG. 4A shows a multiplex IF image of a tissue specimen labeled with the counterstain 4′-6-diamidino-2-phenylindole (DAPI) and the biomarker cytokeratin 18 (CK18), which bind to target proteins in nuclei and cytoplasm, respectively. Although the original image was a pseudo-color image generally exhibiting blue and green corresponding to DAPI and CK18, respectively, the image is provided in gray-scale in FIG. 4A for ease of reproducibility. FIG. 4B shows the image in FIG. 4A segmented into epithelial nuclei (EN) 402, cytoplasm 404, and stroma nuclei 406. Although in the original, segmented and classified image the segmented EN 402 are shown in blue, the segmented cytoplasm 404 are shown in green, and the segmented stroma nuclei 406 are shown in purple, the image is provided in gray-scale in FIG. 4B for ease of reproducibility.

In some embodiments of the present invention, as an alternative to or in addition to the molecular features which are measured in digitized images of tissue subject to multiplex IF, one or more morphometric features may be measured in the IF images. IF morphometric features represent data extracted from basic relevant histologic objects and/or from graphical representations of binary images generated from, for example, a specific segmented view of an object class (e.g., a segmented epithelial nuclei view may be used to generate minimum spanning tree (MST) features). Additional details regarding MST features are described in above-incorporated, commonly-owned U.S. Pub. No. 20100184093. Because of its highly specific identification of molecular components and consequent accurate delineation of tissue compartments—as compared to the stains used in light microscopy—multiplex IF microscopy offers the advantage of more reliable and accurate image segmentation. In some embodiments of the present invention, multiplex IF microscopy may replace light microscopy altogether. In other words, in some embodiments (e.g., depending on the medical condition under consideration), all morphometric and molecular features may be measured through IF image analysis thus eliminating the need for, for example, H&E staining (e.g., some or all of the features listed in Tables 1 and 2 above-incorporated, commonly-owned U.S. Pub. No. 20100184093 could be measured through IF image analysis).

In an immunofluorescence (IF) image, objects are defined by identifying an area of fluorescent staining above a threshold and then, where appropriate, applying shape parameters and neighborhood restrictions to refine specific object classes. In some embodiments, the relevant morphometric IF object classes include epithelial objects (objects positive for cytokeratin 18 (CK18)) and complementary epithelial nuclei (DAPI objects in spatial association with CK18). Specifically, for IF images, the process of deconstructing the image into its component parts is the result of expert thresholding (namely, assignment of the ‘positive’ signal vs. background) coupled with an iterative process employing machine learning techniques. The ratio of biomarker signal to background noise is determined through a process of intensity thresholding. For the purposes of accurate biomarker assignment and subsequent feature generation, supervised learning is used to model the intensity threshold for signal discrimination as a function of image background statistics. This process is utilized for the initial determination of accurate DAPI identification of nuclei and then subsequent accurate segmentation and classification of DAPI objects as discrete nuclei. A similar process is applied to capture and identify a maximal number of CK18+ epithelial cells, which is critical for associating and defining a marker with a specific cellular compartment. These approaches are then applied to the specific markers of interest, resulting in feature generation which reflects both intensity-based and area-based attributes of the relevant protein under study. Additional details regarding this approach, including sub-cellular compartment co-localization strategies, are described in above-incorporated PCT Application No. PCT/US2008/004523, filed Apr. 7, 2008. Additional details regarding multiplex IF image segmentation are also described in above-incorporated, commonly-owned U.S. Pub. No. 20100184093.

### EXAMPLES

### Predicting Disease Progression Post-Radiotherapy

Two new models were developed in accordance with embodiments of the present invention. As described in greater detail below, model 1 contained the biopsy Gleason score (BGS), PSA and two H&E morphometric features with a predictive accuracy concordance index (CI) of 0.86, sensitivity 0.83 and specificity 0.88. Model 2 was developed without clinical variables and contained one morphometric feature and one molecular immunofluorescence (IF) feature, i.e., the relative area of Ki67 positive tumor epithelial nuclei. Model 2 performed with a CI 0.82, sensitivity 0.75 and specificity 0.84. In addition, a prior pretreatment biopsy model (described in above-incorporated, commonly-owned U.S. Pub. No. 20100184093 in connection with FIG. 11 previously generated to predict disease progression in, for example, disease progression in patients treated with radical prostatectomy and followed for a median of 8 years) performed with a CI 0.79, sensitivity 0.91 and specificity 0.60, for predicting disease progression within 8 years on the same data.

Methods: Disease progression was defined as castrate PSA rise, systemic metastasis, and/or death of disease. 52 patients from a 72 EBRT cohort had complete clinical, morphometric and immunofluorescence (IF) biomarker feature data for inclusion in multivariate models. The mean age was 68 yrs, mean PSA 14.31, 36% biopsy Gleason score (BGS)<=6, 40% BGS 7 and 67% T1c. A demographics summary is provided in Table 1 below. Biopsy H&E morphometry, and quantitative IF biomarker data was generated as previously described (Donovan et al., J Urol., 2009; see also above-incorporated, commonly-owned U.S. Pub. Nos. 20100177950 and 20100184093). Performance was evaluated based on the concordance index (CI), sensitivity and specificity.

TABLE 1
Cohort Demographics Summary

| | | |
|---|---|---|
| Total Number: | 52 | |
| Events: | 14 | 27% |
| Sens/Spec Patients | 38 | |
| High Risk | 12 | 32% |
| Low Risk | 38 | 68% |
| Mean Age | 67.8 | |
| PSA | | |
| PSA < 5 | 6 | |
| 5 <= PSA < 10 | 19 | |
| 10 <= PSA < 15 | 11 | |
| 15 <= PSA < 20 | 4 | |
| PSA >= 20 | 12 | |
| Mean PSA | 14.31 | |
| Dominant Biopsy Gleason | | |
| Dominant Gleason 1 | 0 | 0.00% |
| Dominant Gleason 2 | 3 | 5.77% |
| Dominant Gleason 3 | 27 | 51.92% |
| Dominant Gleason 4 | 15 | 28.85% |
| Dominant Gleason 5 | 7 | 13.46% |
| Biopsy Gleason Sum | | |
| Gleason Sum 4 | 1 | 1.92% |
| Gleason Sum 5 | 7 | 13.46% |
| Gleason Sum 6 | 11 | 21.15% |
| Gleason Sum 7 | 21 | 40.38% |
| Gleason Sum 8 | 3 | 5.77% |
| Gleason Sum 9 | 4 | 7.69% |
| Gleason Sum 10 | 5 | 9.62% |
| Stage | | |
| Missing | 1 | 1.92% |
| T1ab | 0 | 0.00% |
| T1c | 35 | 67.31% |
| T2 | 16 | 30.77% |

Model 1: Predicting Disease Progression Post-Radiotherapy Clinical, Molecular, and Morphometric Data

Clinical, morphometric, and molecular data for each external beam radiotherapy (EBRT) patient cohort were analyzed to produce a model that predicts, based on data available at the time of diagnosis of prostate cancer in a patient, the likelihood of disease progression in the patient even if the patient is treated with primary radiotherapy. Aureon's proprietary SVRc was used to build the model (see e.g., above-incorporated, commonly-owned U.S. Pat. No. 7,505,948). Two clinical features and two morphometric features were selected for the final model. In this embodiment, no molecular features were selected. The morphometric features were measured from digital images of H&E-stained tissue. In other embodiments, these features and/or other clinical, molecular, and/or morphometric features (e.g., one or more of the features disclosed in commonly-owned, above-incorporated U.S. Pub. Nos. 20100177950 and 20100184093) may be included in a final model that is predictive of disease progression post-radiotherapy. In other embodiments, some or all of the morphometric features included in the model may be measured from digital images of tissue subject to multiplex quantitative immunofluorescence (IF). The clinical and morphometric features selected for inclusion in this model are listed in FIG. 5 and described in Table 2 below:

TABLE 2
Features Selected for Inclusion in Model 1

| Feature | Weight in Final Model | Feature Description |
|---|---|---|
| ‘HE02_Lum_Are_Median’ | 8.4411 | Median area of lumens (morphometric) |
| ‘bxgscore’ | −33.6667 | Biopsy Gleason Score (clinical) |
| ‘preop_psa’ | −30.4439 | Preoperative PSA (clinical) |
| ‘HEx2_nta_EpiIsoNuc_Are_Tot’ | −20.9061 | Relative area of epithelial nuclei relative |
| | | to total tumor area outlined or otherwise |
| | | identified (morphometric) |

Table 3 below lists performance metrics for Model 1. It also lists the features selected and removed during forward and backward feature selection and their effects on the CI. In other embodiments, some or all of the features removed during backward feature selection (two morphometric features and one molecular feature) and/or other features may be included in a final model (e.g., Model 1) that predicts, based on data available at the time of diagnosis of prostate cancer in a patient, the likelihood of disease progression in the patient even if the patient is treated with primary radiotherapy.

TABLE 3
Performance Metrics and Feature Selection for Model 1

| Training CI on complete dataset: | 0.8604 |
|---|---|
| Training Sensitivity/Specificity | 42.7036 |
| Threshold: | |
| Train Sensitivity: | 0.8333 |
| Train Specificity: | 0.8846 |
| Features Chosen | CI after Adding Feature |
| HE02_Lum_Are_Median | 0.797978 |
| bxgscore | 0.832930 |
| HEx2_RelArea_EpiNucCyt_Lum | 0.834150 |
| IFx2_RelAreEN_Ki67p_Area2EN | 0.837321 |
| HEx2_RelArea_Cyt_Out2WinGU | 0.838468 |
| preop_psa | 0.843710 |
| HEx2_nta_EpiIsoNuc_Are_Tot | 0.846369 |
| Backward feature selection | |
| IFx2_RelAreEN_Ki67p_Area2EN - | 0.851874 |
| OUT | |
| HEx2_RelArea_EpiNucCyt_Lum - | 0.852941 |
| OUT | |
| HEx2_RelArea_Cyt_Out2WinGU - | 0.852941 |
| OUT | |

Feature “IFx2_RelAreEN_Ki67p_Area2EN” which was removed during backward feature selection in this example is a normalized area molecular feature representing the relative area of Ki67-positive epithelial nuclei to the total area of epithelial nuclei, as observed in digital images of tissue subject to multiplex quantitative IF. Feature “HEx2_RelArea_EpiNucCyt_Lum” is a morphometric feature representing the ratio of the area of epithelial cells (nuclei+cytoplasm) to the area of lumens, as observed in digital images of H&E-stained tissue. Feature “HEx2_RelArea_Cyt_Out2WinGU” is a morphometric feature representing the ratio of the area of epithelial cytoplasm outside of gland units to the area of epithelial cytoplasm within (inside) gland units, as observed in digital images of H&E-stained tissue. Gland units were identified in the tissue images as described above an in above-incorporated, commonly-owned U.S. Pub. Nos. 20100177950 and 20100184093.

Model 2: Predicting Disease Progression Post-Radiotherapy Molecular and Morphometric Data Only

Another model was generated (without use of clinical data) that predicts, based on data available at the time of diagnosis of prostate cancer in a patient, the likelihood of disease progression in the patient even if the patient is treated with primary radiotherapy. Again, Aureon's proprietary SVRc was used to build the model. One morphometric and one molecular feature were selected for the final model. In other embodiments, these features and/or other clinical, molecular, and/or morphometric features (e.g., one or more of the features disclosed in commonly-owned, above-incorporated U.S. Pub. Nos. 20100177950 and 20100184093) may be included in a final model that is predictive of disease progression post-radiotherapy. The morphometric and molecular features selected for inclusion in this model are listed in FIG. 6 and described in Table 4 below:

TABLE 4
Features Selected for Inclusion in Model 2

| Feature | Weight in Final Model | Feature Description |
|---|---|---|
| ‘HE02_Lum_Are_Median’ | 15.107 | Median area of lumens (morphometric) |
| ‘IFx2_RelAreEN_Ki67p_Area2MDT’ | −20.4703 | Relative area of Ki67-positive |
| | | epithelial nuclei to area of tumor as |
| | | defined manually (or otherwise in |
| | | other examples) (molecular) |

Table 5 below lists performance metrics for Model 2. It also lists the features selected during feature selection and their effect on the CI.

TABLE 5
Performance Metrics and Feature Selection for Model 1

| Training CI on complete dataset: | 0.8184 |
|---|---|
| Training Sensitivity/Specificity | 40.8290 |
| Threshold: | |
| Train Sensitivity: | 0.75 |
| Train Specificity: | 0.8462 |
| Features Chosen | CI after Adding Feature |
| HE02_Lum_Are_Median | 0.808538 |
| IFx2_RelAreEN_Ki67p_Area2MDT | 0.828077 |

In addition, and for comparison to Models 1 and 2, a prior pretreatment biopsy model (described in commonly-owned U.S. Pub. No. 20100184093 in connection with FIG. 11) was used to evaluate the same EBRT patient data (52 patients). As summarized in Table 6 below, the existing model performed with a CI 0.79, sensitivity 0.91 and specificity 0.60, for predicting DP within 8 years, thus also demonstrating that it accurately predicted disease progression for patient's post-EBRT.

TABLE 6
Performance Metrics of Existing Disease Progression Model

| | |
|---|---|
| Validation CI: | 0.7935 |
| Validation Sensitivity: | 0.9167 |
| Validation Specificity: | 0.6071 |
| Validation Hazard Ratio: | 17.9818 |
| HR P-Value | 0.0055 |

These values of the sensitivity, specificity, and hazard ratio were calculated by using the existing cutpoint of approximately 30 for this model, as described in above-incorporated, commonly-owned U.S. Pub. No. 20100184093.

In view of the foregoing, it can be seen that models are provided that accurately predict disease progression for patients post-radiation therapy. Such models may evaluate clinical data, molecular data, and/or computer-generated morphometric data generated from one or more tissue images. In addition, in some embodiments, a model constructed without clinical variables.

### Additional Embodiments

Thus it is seen that methods and systems are provided for treating, diagnosing and predicting the occurrence of a medical condition such as, for example, the likelihood of disease progression in a patient even if the patient is treated with primary radiotherapy. Although particular embodiments have been disclosed herein in detail, this has been done by way of example for purposes of illustration only, and is not intended to be limiting with respect to the scope of the appended claims, which follow. In particular, it is contemplated by the present inventors that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The present inventors reserve the right to pursue such inventions in later claims.

Insofar as embodiments of the invention described above are implementable, at least in part, using a computer system, it will be appreciated that a computer program for implementing at least part of the described methods and/or the described systems is envisaged as an aspect of the present invention. The computer system may be any suitable apparatus, system or device. For example, the computer system may be a programmable data processing apparatus, a general purpose computer, a Digital Signal Processor or a microprocessor. The computer program may be embodied as source code and undergo compilation for implementation on a computer, or may be embodied as object code, for example.

It is also conceivable that some or all of the functionality ascribed to the computer program or computer system aforementioned may be implemented in hardware, for example by means of one or more application specific integrated circuits.

Suitably, the computer program can be stored on a carrier medium in computer usable form, which is also envisaged as an aspect of the present invention. For example, the carrier medium may be solid-state memory, optical or magneto-optical memory such as a readable and/or writable disk for example a compact disk (CD) or a digital versatile disk (DVD), or magnetic memory such as disc or tape, and the computer system can utilize the program to configure it for operation. The computer program may also be supplied from a remote source embodied in a carrier medium such as an electronic signal, including a radio frequency carrier wave or an optical carrier wave.

All of the following commonly-owned disclosures are hereby incorporated by reference herein in their entireties: U.S. application Ser. No. 12/462,041, filed on Jul. 27, 2009; U.S. application Ser. No. 12/584,048, filed Aug. 28, 2009; PCT Application No. PCT/US09/04364, filed on Jul. 27, 2009; PCT Application No. PCT/US08/004523, filed Apr. 7, 2008, which claims priority from U.S. Provisional Patent Application Nos. 60/922,163, filed Apr. 5, 2007, 60/922,149, filed Apr. 5, 2007, 60/923,447, filed Apr. 13, 2007, and 61/010,598, filed Jan. 9, 2008; U.S. patent application Ser. No. 11/200,758, filed Aug. 9, 2005 (now U.S. Pat. No. 7,761,240); U.S. patent application Ser. No. 11/581,043, filed Oct. 13, 2006; U.S. patent application Ser. No. 11/404,272, filed Apr. 14, 2006; U.S. patent application Ser. No. 11/581,052, filed Oct. 13, 2006 (now U.S. Pat. No. 7,461,048), which claims priority from U.S. Provisional Patent Application No. 60/726,809, filed Oct. 13, 2005; U.S. patent application Ser. No. 11/080,360, filed Mar. 14, 2005 (now U.S. Pat. No. 7,467,119); U.S. patent application Ser. No. 11/067,066, filed Feb. 25, 2005 (now U.S. Pat. No. 7,321,881), which claims priority from U.S. Provisional Patent Application Nos. 60/548,322, filed Feb. 27, 2004, and 60/577,051, filed Jun. 4, 2004; U.S. patent application Ser. No. 10/991,897, filed Nov. 17, 2004 (now U.S. Pat. No. 7,483,554), which claims priority from U.S. Provisional Patent Application No. 60/520,815, filed Nov. 17, 2003; U.S. patent application Ser. No. 10/624,233, filed Jul. 21, 2003 (now U.S. Pat. No. 6,995,020, issued Feb. 7, 2006); U.S. patent application Ser. No. 10/991,240, filed Nov. 17, 2004 (now U.S. Pat. No. 7,505,948), which claims priority from U.S. Provisional Patent Application No. 60/520,939 filed Nov. 18, 2003; and U.S. Provisional Patent Application Nos. 60/552,497, filed Mar. 12, 2004, 60/577,051, filed Jun. 4, 2004, 60/600,764, filed Aug. 11, 2004, 60/620,514, filed Oct. 20, 2004, 60/645,158, filed Jan. 18, 2005, and 60/651,779, filed Feb. 9, 2005.

What is claimed is:

1. Apparatus for predicting disease progression in a patient post-radiation therapy, the apparatus comprising:
a model predictive of progression of the disease post-radiation therapy configured to evaluate a dataset for a patient to produce a value indicative of a whether the disease is likely to progress in the patient after radiation therapy, wherein the model is based on one or more clinical features, one or more molecular features, and/or one or more computer-generated morphometric feature(s) generated from one or more tissue image(s).

2. The apparatus of claim 1, wherein the model is predictive of progression of prostate cancer.

3. The apparatus of claim 1, wherein the model is based on said one or more clinical features, said one or more molecular features, and said one or more computer-generated morphometric feature(s) generated from one or more tissue image(s).

4. The apparatus of claim 3, wherein said one or more molecular features and said one or more computer-generated morphometric features are generated from a needle biopsy of tissue taken from the patient at diagnosis before treatment of the patent with said radiation therapy.

5. The apparatus of claim 3, wherein at least one of said one or more computer-generated morphometric features is generated from computer analysis of one or more images of tissue subject to staining with hematoxylin and eosin (H&E).

6. The apparatus of claim 3, wherein at least one of said one or more computer-generated morphometric features or said one or more molecular features is generated from computer analysis of one or more images of tissue subject to multiplex immunofluorescence (IF).

7. The apparatus of claim 1, wherein the model is based on one or more of the following features:
pre-operative PSA;
Gleason score;
a morphometric measurement of lumens derived from a tissue image; and
a morphometric measurement of epithelial nuclei derived from a tissue image.

8. The apparatus of claim 7, wherein said morphometric measurement of lumens comprises a median area of lumens.

9. The apparatus of claim 7, wherein said morphometric measurement of epithelial nuclei comprises the relative area of epithelial nuclei relative to total tumor area.

10. The apparatus of claim 7, wherein the model is based on all of said features listed in claim 7.

11. The apparatus of claim 7, wherein the model is further based on one or more additional clinical, molecular, and/or morphometric features.

12. The apparatus of claim 1, wherein the model is based on at least on a molecular feature representing the relative area of Ki67-positive epithelial nuclei to the total area of epithelial nuclei.

13. The apparatus of claim 1, wherein the model is based on one or more of the following features:
a morphometric measurement of lumens derived from a tissue image; and
a molecular measurement of Ki67-positive epithelial nuclei.

14. The apparatus of claim 13, wherein said molecular measurement of Ki67-positive epithelial nuclei comprises the relative area of Ki67-positive epithelial nuclei to area of tumor.

15. The apparatus of claim 13, wherein the model is based on both of said features listed in claim 12.

16. The apparatus of claim 1, wherein the model is not based on any clinical features.

17. A method of predicting disease progression in a patient post-radiation therapy, the method comprising:
evaluating a dataset for a patient with a model predictive of progression of the disease post-radiation therapy, wherein the model is based on one or more clinical features, one or more molecular features, and/or one or more computer-generated morphometric feature(s) generated from one or more tissue image(s),
thereby evaluating whether the disease is likely to progress in the patient after radiation therapy.

18. The method of claim 17, wherein the model is predictive of progression of prostate cancer.

19. The method of claim 17, wherein the model is based on said one or more clinical features, said one or more molecular features, and said one or more computer-generated morphometric feature(s) generated from one or more tissue image(s).

20. The method of claim 19, further comprising generating said one or more molecular features and said one or more computer-generated morphometric features from a needle biopsy of tissue taken from the patient at diagnosis before treatment of the patent with said radiation therapy.

21. Computer-readable media having computer program instructions recorded thereon for causing a computer to perform the method comprising:
evaluating a dataset for a patient with a model predictive of progression of the disease post-radiation therapy, wherein the model is based on one or more clinical features, one or more molecular features, and one or more computer-generated morphometric feature(s) generated from one or more tissue image(s),
thereby evaluating whether the disease is likely to progress in the patient after radiation therapy.

22. Apparatus for predicting disease progression in a patient post-radiation therapy, the apparatus comprising:
means for evaluating a dataset for a patient with a model predictive of progression of the disease post-radiation therapy, wherein the model is based on one or more clinical features, one or more molecular features, and one or more computer-generated morphometric feature(s) generated from one or more tissue image(s),
thereby evaluating whether the disease is likely to progress in the patient after radiation therapy.
